(19) United States
(12) Patent Application Publication
(10) Pub. No.: US 2022/0378286 A1
(43) Pub. Date: Dec. 1, 2022

(54) VAGINAL EXAMINATION DEVICE AND METHODS OF USE THEREOF

(71) Applicant: Virginia Commonwealth University, Richmond, VA (US)

(72) Inventor: Ramesh C. Seeras, South Hill, VA (US)

(21) Appl. No.: 17/752,894

(22) Filed: May 25, 2022

Related U.S. Application Data
(60) Provisional application No. 63/193,277, filed on May 26, 2021.

(51) Int. Cl. A61B 1/303; A61B 1/00; A61B 1/32; A61B 1/05

(52) U.S. Cl. CPC A61B 1/303; A61B 1/00016; A61B 1/32; A61B 1/05; A61B 1/00071

(57) ABSTRACT

A vaginal examination device includes a hollow cylindrical body having a distal end and a proximal end, wherein the hollow body is partitioned into two or more channels; one or more inner channels, wherein the inner channels allow insertion and removal of examination modules; one or more extendable blades, wherein the blades are attached to the distal end of the body; one or more levers at the proximal end of the body configured to actuate the one or more extendable blades; and one or more handles at the proximal end of the body. Also provided are methods of self-performing pelvic examination using a vaginal examination device.

### CROSS-REFERENCE TO RELATED APPLICATIONS

This application claims the benefit of U.S. provisional patent application 63/193,277, filed May 26, 2021, the complete contents of which is hereby incorporated by reference.

### FIELD OF THE INVENTION

The invention relates to a medical device for examining a resilient cavity. In particular, the invention provides a vaginoscope for pelvic examination and/or specimen collection which may be self-inserted by the user.

### BACKGROUND OF THE INVENTION

Typical recommendations include a minimum of one pelvic examination per year for a female during her reproductive years. During a pelvic examination, a clinician views the interior genitalia of the female patient while the female patient is in a supine with her feet in the stirrups of an examination table. A vaginal speculum is the medical device used for examination of the vaginal wall and cervix and collection of specimens for laboratory tests. The speculum is inserted into the vagina to dilate it for examination of the vagina and cervix and to allow access to the uterus. This process causes discomfort, embarrassment, and a feeling of vulnerability in many patients.

Thus, there is a need to develop novel devices and methods so that a female patient herself, without the assistance of a clinician, enable visualization of the vagina and cervix and allow for the collection of specimens.

### SUMMARY

Embodiments of the disclosure provide a medical diagnostic device for examining a resilient cavity. The device is designed for pelvic examination and/or vaginal specimen collection for laboratory tests. A vaginal examination device, according to embodiments of the disclosure, is a device that can be self-inserted by a patient thus leading to a reduction in discomfort, embarrassment, and sense of vulnerability.

An aspect of the disclosure provides a vaginal examination device comprising a hollow cylindrical body having a distal end and a proximal end, wherein the hollow cylindrical body is partitioned into two or more channels; one or more inner channels, wherein the inner channels allow insertion and removal of examination modules; one or more extendable blades, wherein the blades are attached to the distal end of the body; one or more levers at the proximal end of the body configured to actuate the one or more extendable blades; and one or more handles at the proximal end of the body.

In some embodiments, the examination modules are selected from the group consisting of light, operative, sampling, and visibility equipment. In some embodiments, the equipment transmits and receives wireless communication. In some embodiments, the device has four extendable blades. In some embodiments, the device has a total length of about 4-6 cm.

Another aspect of the disclosure provides a method for self-performing pelvic examination comprising at least the following steps: inserting the distal end of a device as described herein into a user's vagina by the user; placing or securing the device within the user's vagina by pulling one or more levers to extend one or more blades outwards; inserting and removing one or more examination modules through the one or more inner channels; pulling the one or more levers to collapse the one or more blades; and removing the device from the user's vagina. In some embodiments, the method further comprises steps of: obtaining photo and/or video information from video camera means arranged on the distal end of the device; and transmitting the photo and/or video information by a wireless communication means to a monitor.

Additional features and advantages of the invention will be set forth in the description below, and in part will be apparent from the description, or may be learned by practice of the invention. The advantages of the invention can be realized and attained by the exemplary structure particularly pointed out in the written description and claims hereof as well as the appended drawings.

### BRIEF DESCRIPTION OF THE DRAWINGS

FIG. 1. View of a vaginal examination device according to some embodiments of the disclosure.

FIG. 2. View of a hollow cylindrical body according to some embodiments of the disclosure.

FIG. 3. View of extendable blades according to some embodiments of the disclosure.

FIG. 4. View of a central ring according to some embodiments of the disclosure.

FIG. 5. View of a hollow cylindrical body according to some embodiments of the disclosure.

FIG. 6. Cross-section view of a vaginal examination device according to some embodiments of the disclosure.

FIG. 7. Alternative cross-section view of a vaginal examination device according to some embodiments of the disclosure.

FIG. 8. View of a lever according to some embodiments of the disclosure.

FIGS. 9A-B. View of a vaginal examination device in a (A) closed and (B) open position according to some embodiments of the disclosure.

### DETAILED DESCRIPTION

Embodiments of the disclosure provide a hand-held vaginal examination device which a woman may self-insert to conveniently view and/or record video images of the interior of her vagina and cervix on a remote monitor, e.g. a computer monitor, television, tablet, or smartphone and to collect specimen samples. The device provides a vaginal speculum comprising a hollow cylindrical body and having means integral therewith for distending the vagina internally of the introitus in use, whereby the speculum allows visualization of, and access to and through the cervix.

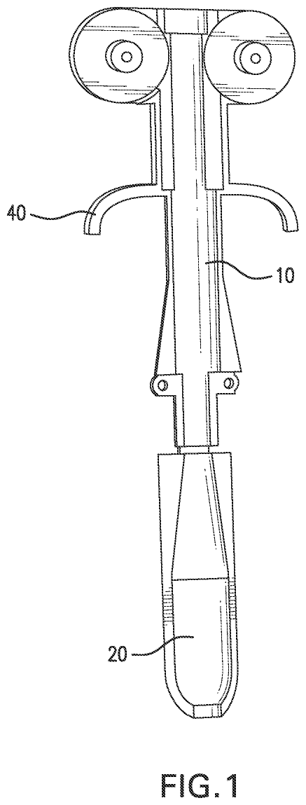

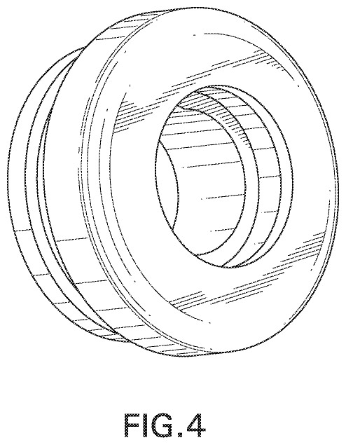

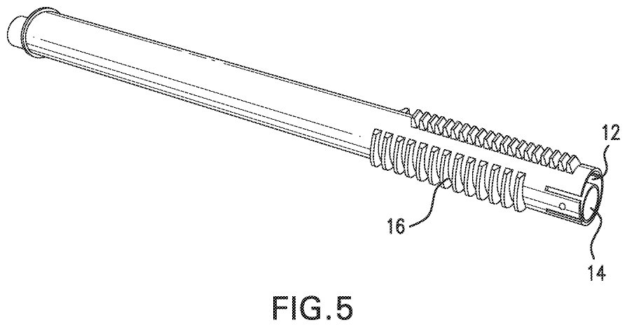

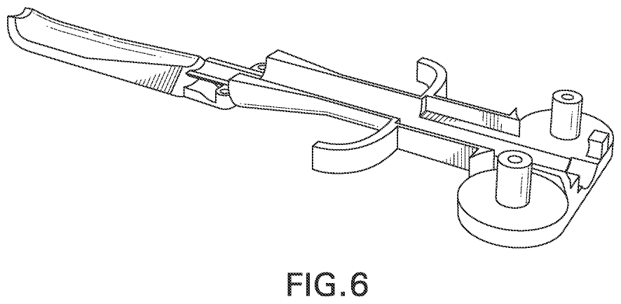

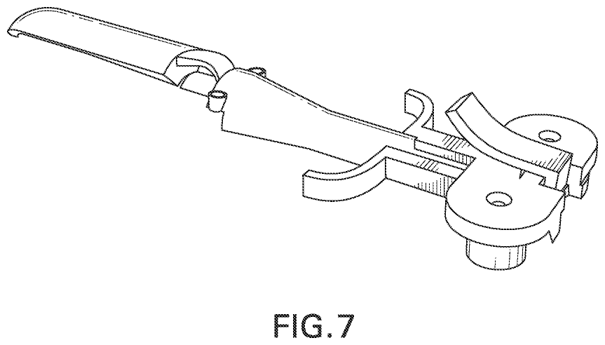

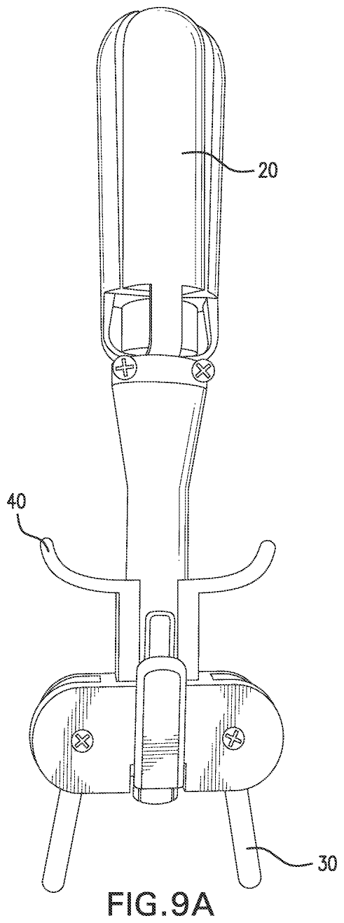

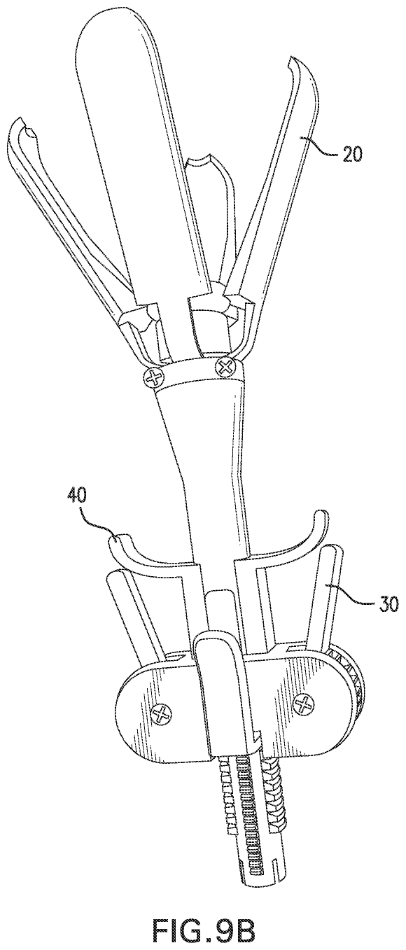

With reference to FIGS. 1 and 5-7, the device comprises a hollow cylindrical body 10 having a distal end and a proximal end, wherein the hollow cylindrical body is partitioned into two or more channels 12. The device also includes one or more inner channels 14 configured for insertion and removal of examination modules. At the distal end of the body 10 are one or more extendable blades 20. With reference to FIGS. 9A-B, at the proximal end of the body are one or more levers 30 configured to actuate the one or more extendable blades 20 and one or more handles 40 to be gripped by the user. The blades 20 may have a bull-nosed tip so that the device can go gently through the introitus in use. The blades 20 may be attached to the body 10 via a central ring (FIG. 4).

The device may be molded from plastic, e.g. a clear or transparent plastic, and can be disposable. In some embodiments, the device is made of one or more biocompatible materials including, but not limited to: metals, polymers (including, but not limited to: nylon, PEBAX, Teflon, polyethylene, and polypropylene), glass, and ceramics.

The inner channels facilitate the use of various instruments such as light, operative, sampling, and visibility equipment which are known in the art. The working devices can be used to perform a variety of diagnostic, therapeutic, or preventive procedures. For example, the inner channels may be used for insertion of cotton swabs, wooden sticks, brushes, and/or tweezers to collect samples for diagnostic screening. The inner channels may also be used to introduce and/or remove one or more devices (e.g. an IUD) and/or fluids into or from the vagina to perform one or more medical procedures. In some embodiments, the inner channels may be used for insertion of surgical instruments such as a biopsy sampler or laser electrode.

The light equipment in any of the embodiments disclosed herein may be a multi-spectral light source or a mono-chromatic light source. The distal region of the device may comprise a diffuser to diffuse light from a light source. In one embodiment, the source of light is within an endoscope. In another embodiment, the source of light is separate from an endoscope. The light source may be integral to a wall of any of the devices disclosed herein.

Examples of visualization modalities include endoscopes and body insertable ultrasound imaging devices. Other visibility equipment which may be utilized include, e.g. a wi-fi enabled camera for taking images or video of the interior of the vagina and the cervix. The camera may include a light source. The camera may wirelessly transmit images and/or video to a remote display, such as a smartphone, tablet, computer monitor, television, or other screen.

Additional operative equipment may include one or more sensors for measuring one or more of the following quantities: temperature, pressure, and sound. A sensor may also provide measurements of the relative amount of blood flow in the wall of the vagina using doppler shifted acoustic measurements, and other blood flow measurement techniques known in the ultrasound art. Some sensors may be used to provide measurements on the fluids within the vagina including the pH; the salinity; the types and quantities of hormones present; the quantity of any blood present; the types and quantities of any bacteria present; the types and quantities of any viruses present; the types and quantities of any fungi present; and perhaps the presence or absence of sperm. A sensor may also provide measurements of the electrical and electrochemical signals generated by the interior of the vagina. Such biosensors are known in the art.

The device as described herein is small, e.g. about the size of a tampon. In some embodiments, the length of the device from the distal end to the proximal end of the body is about 3-10 cm, e.g. about 4-6 cm. The size of the device as described herein may be configured to accommodate various patient vessel sizes, ages, or surgical procedures.

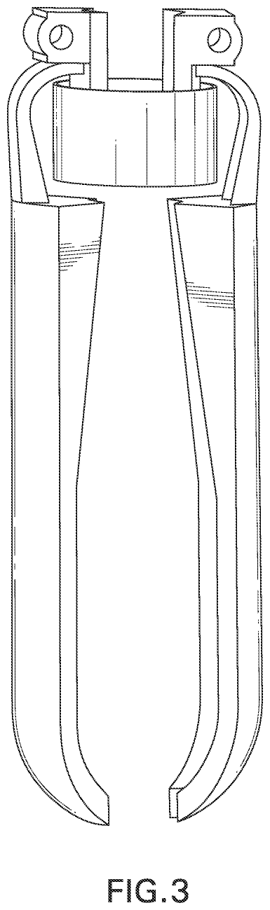

With reference to FIGS. 1 and 3, the device includes moveable blades 20 disposed circumferentially about the longitudinal axis of the body 10. In some embodiments, the device may include 2-6 blades, e.g. 4 blades.

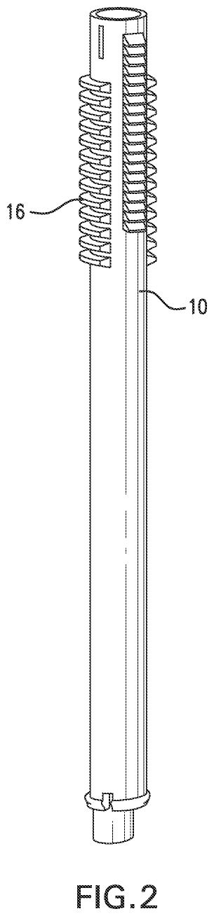

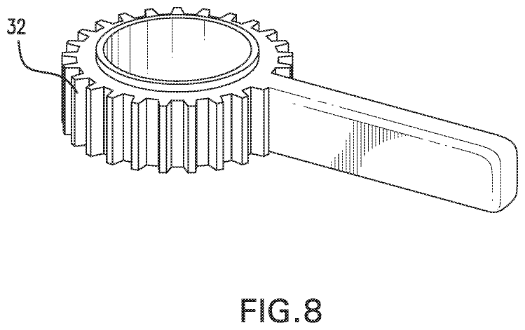

With reference to FIGS. 9A-B, actuation of the levers 30 cause the blades 20 to open. As shown in FIGS. 2, 5, and 8, both the body 10 and the levers 30 have ridges 16 and 32, respectively. As the lever is turned, the ridges 32 on the levers 30 engage with subsequent ridges 16 of the body 10, thus moving the body 10 and opening the blades 20. In some embodiments, the blades 20, levers 30, and handles 40 form an outer tube around the hollow cylindrical body 10 which are movable relative to each other along the longitudinal axis of the hollow cylindrical body 10 between a first position, wherein the blades 20 are closed and a second position, wherein the blades 20 are extended. The disposition of the blades 20 means that when they are extended in the second position they will distend the vagina on all sides.

Embodiments of the disclosure provide methods for self-performing pelvic examination comprising at least the following steps: inserting the distal end of a device as described herein into a user's vagina by the user; placing or securing the device within the user's vagina by pulling one or more levers to extend one or more blades outwards; inserting and removing one or more examination modules through the one or more inner channels; pulling the one or more levers to collapse the one or more blades; and removing the device from the user's vagina. Thus, the device is inserted through the introitus into the vagina with the hinged blades folded together. Once positioned in the vagina the blades are moved apart by manipulation of a lever so as to dilate the vagina, in order to facilitate access to and examination of the vagina and cervix.

In some embodiments, the method further comprises steps of: obtaining photo and/or video information from video camera means arranged on the distal end of the device; and transmitting the photo and/or video information by a wireless communication means to a monitor as described herein.

Surgical and clinical applications of the device include, but are not limited to, detection of cervical cancer and pre-cancer e.g. via cytology and colposcopy. The device may also be used in a standard gynecological exam which involves visual examination of the vaginal interior cavity and related structures, which may optionally include the use of magnification, pulpation of the pelvic region and a Pap smear. When conducting Pap smear screenings, the clinician gently scrapes and collects exfoliated cells from the surface of the cervix by a thin wooden stick and a tiny brush and places them on slides that are sent to a cytologist for further examination. If any abnormal areas are seen, the doctor will take a biopsy of the tissue. In a biopsy, a tiny sample of tissue will be removed from the area with a tweezer-like instrument. An endocervical scraping from the os (the opening in the middle of the cervix) may be taken as this is the area where often abnormal cells begin.

It is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

While the invention has been described in terms of its preferred embodiments, those skilled in the art will recognize that the invention can be practiced with modification within the spirit and scope of the appended claims. Accordingly, the present invention should not be limited to the embodiments as described above, but should further include all modifications and equivalents thereof within the spirit and scope of the description provided herein.

We claim:

1. A vaginal examination device comprising:
a hollow cylindrical body having a distal end and a proximal end, wherein the hollow cylindrical body is partitioned into two or more channels;
one or more inner channels, wherein the inner channels allow insertion and removal of examination modules;
one or more extendable blades, wherein the blades are attached to the distal end of the body;
one or more levers at the proximal end of the body configured to actuate the one or more extendable blades; and
one or more handles at the proximal end of the body.

2. The vaginal examination device according to claim 1, wherein the examination modules are selected from the group consisting of light, operative, sampling, and visibility equipment.

3. The vaginal examination device according to claim 2, wherein the equipment transmits and receives wireless communication.

4. The vaginal examination device according to claim 1, wherein the device has four extendable blades.

5. The vaginal examination device according to claim 1, wherein the device has a total length of about 4-6 cm.

6. A method for self-performing pelvic examination comprising at least the following steps:
inserting the distal end of the device of claim 1 into a user's vagina by the user;
placing or securing the device within the user's vagina by pulling one or more levers to extend one or more blades outwards;
inserting and removing one or more examination modules through the one or more inner channels;
pulling the one or more levers to collapse the one or more blades; and
removing the device from the user's vagina.

7. The method of claim 6, wherein the examination modules are selected from the group consisting of light, operative, sampling, and visibility equipment.

8. The method of claim 6, further comprising steps of:
obtaining photo and/or video information from video camera means arranged on the distal end of the device; and
transmitting the photo and/or video information by a wireless communication means to a monitor.
